(19) United States
(12) Patent Application Publication
(10) Pub. No.: US 2015/0031882 A1
(43) Pub. Date: Jan. 29, 2015

(54) METHODS FOR TREATING CANCER USING PI3K INHIBITOR AND MEK INHIBITOR

(71) Applicants: SANOFI, Paris (FR); MERCK PATENT GmbH, Darmstadt (DE)

(72) Inventors: Karl HSU, Lexington, MA (US); Joanne LAGER, Hollis, NH (US); Janet OGDEN, Westwood, MA (US)

(21) Appl. No.: 14/504,970

(22) Filed: Oct. 2, 2014

Related U.S. Application Data
(63) Continuation of application No. PCT/US2013/035231, filed on Apr. 4, 2013.
(60) Provisional application No. 61/771,457, filed on Mar. 1, 2013.
(60) Provisional application No. 61/621,252, filed on Apr. 6, 2012.

(30) Foreign Application Priority Data
Feb. 12, 2013 (FR) 1351158

(51) Int. Cl. A61K 31/519; A61K 31/44

(52) U.S. Cl. CPC A61K 31/519; A61K 31/44
USPC 544/279

(57) ABSTRACT

Methods of treating patients with cancer are provided, wherein the methods comprise administering to the patient an effective amount of a MEK inhibitor and an effective amount of a PI3K inhibitor. Compositions in which the MEK and PI3K inhibitors are combined also are described.

### BACKGROUND

There is an ongoing need in the art for more efficacious methods and compositions in the treatment of cancer. The instant application is directed, generally, to compositions and methods for the treatment of cancer, and more particularly, to compositions and methods comprising inhibitors of the mitogen activated protein kinase (MEK) and/or phosphoinositide 3-kinase (PI3K) pathways.

Tumor cells treated with inhibitors of MEK kinases typically respond via inhibition of phosphorylation of ERK, down-regulation of Cyclin D, induction of G1 arrest, and finally undergoing apoptosis. Pharmacologically, MEK inhibition completely abrogates tumor growth in BRaf xenograft tumors whereas Ras mutant tumors exhibit only partial inhibition in most cases (D. B. Solit et al., Nature 2006; 439: 358-362). Thus, MEKs have been targets of great interest for the development of cancer therapeutics.

N—((S)-2,3-dihydroxypropyl)-3-(2-fluoro-4-iodo-phenylamino)isonicotinamide, referred to herein as “Compound (1)”, is a novel, allosteric inhibitor of MEK. It possesses relatively high potency and selectivity, having no activity against 217 kinases or 90 non-kinase targets when tested at 10 μM. The in vivo PK profile of Compound (1) is acceptable in mice and rats, with relatively high oral bioavailability (52-57%), medium or high clearance (0.9-2.6 L/h/kg) and medium or long half-life (2.2-4.7 h).

2-amino-8-ethyl-4-methyl-6-(1H-pyrazol-5-yl)pyrido[2,3-d]pyrimidin-7(8H)-one, referred to herein as “Compound (2)”, is a selective inhibitor of class I PI3K lipid kinases. Compound (2) targets both PI3K isoforms (IC₅₀ values in nM: PI3Kα39, PI3Kβ113, PI3Kδ43, PI3Kγ9) and mTOR (157 nM). Oral administration of Compound (2) alone inhibits tumor growth in mice bearing xenografts in which PI3K signaling is activated, such as the PTEN-deficient PC-3 prostate adenocarcinoma, U87-MG gliobastoma, A2058 melanoma and WM-266-4 melanoma, or the PIK3CA mutated MCF7 mammary carcinoma. Compound (2) is currently undergoing testing in Phase I clinical trials for patients with solid tumor, lymphoma or glioblastoma and in a Phase I/II trial for patients with hormone receptor-positive breast cancer.

There remains a need, however, for a cancer therapy that is more effective in inhibiting cell proliferation and tumor growth while minimizing patient toxicity. There is a particular need for an MEK or PI3K inhibitor therapy which is more efficacious without substantially increasing, or even maintaining or decreasing, the dosages of MEK or PI3K inhibitor traditionally employed in the art.

### SUMMARY

In one aspect, there are provided compositions and uses thereof in the treatment of a variety of cancers.

In one embodiment, a method of treating cancer in a human patient comprises administering to the patient an effective amount of (a) 2-amino-8-ethyl-4-methyl-6-(1H-pyrazol-5-yl)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, wherein said cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

In another embodiment, a method of treating cancer in a human patient comprises administering to the patient an effective amount of (a) 2-amino-8-ethyl-4-methyl-6-(1H-pyrazol-5-yl)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, wherein said cancer is recurrent low grade serous ovarian cancer. In one aspect, the treatment is administered after at least one prior line of systemic therapy.

In some embodiments, the cancer is relapsed or refractory.

In some embodiments, the method comprises at least one cycle, wherein the cycle is a period of 3 weeks, wherein for each cycle the 2-amino-8-ethyl-4-methyl-6-(1H-pyrazol-5-y1)pyrido[2,3-d]pyrimidin-7(8H)-one or pharmaceutically acceptable salt thereof is administered at a daily dose of about 30, 50, 70 or 90 mg and the N—((S)-2,3-dihydroxypropyl)-3-(2-fluoro-4-iodo-phenylamino)isonicotinamide or pharmaceutically acceptable salt thereof is administered at a daily dose of about 15, 30, 60 or 90 mg. 5. In one embodiment, for each cycle the 2-amino-8-ethyl-4-methyl-6-(1H-pyrazol-5-yl)pyrido[2,3-d]pyrimidin-7(8H)-one or pharmaceutically acceptable salt thereof is administered at a daily dose of about 70 mg and the N—((S)-2,3-dihydroxypropyl)-3-(2-fluoro-4-iodo-phenylamino)isonicotinamide or pharmaceutically acceptable salt thereof is administered at a daily dose of about 60 mg.

In some aspects, the effective amount in the claimed methods produces at least one therapeutic effect selected from the group consisting of reduction in size of a tumor, reduction in metastasis, complete remission, partial remission, stable disease, increase in overall response rate, or a pathologic complete response. In others, the effective amount achieves a synergistic effect in reducing a tumor volume in said patient. In still others, the effective amount achieves tumor stasis in said patient. In other aspects, the effective amount is clinically proven safe.

In another aspect, compositions are provided for use in treating cancer in a human patient, the composition comprising an effective amount (a) 2-amino-8-ethyl-4-methyl-6-(1H-pyrazol-5-yl)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, wherein said cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

In other aspects, uses of a combination are provded that comprise a therapeutically effective amount of (a) 2-amino-8-ethyl-4-methyl-6-(1H-pyrazol-5-yl)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, for the preparation of a medicament for use in treatment of cancer, wherein said cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

In another aspect, kits are provided comprising: (A) the compound of Formula (1), or a pharmaceutically acceptable salt thereof; (B) the compound of Formula (2), or a pharmaceutically acceptable salt thereof; and (C) instructions for use.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 graphically presents the antitumor activity of Compound (2) and Compound (3) as single agents and in combination with Compound (1) against SCID female mice bearing human CRC CR-IC-0013M patient-derived xenografts.

FIG. 2 graphically presents the antitumor activity of Compound (2) and Compound (3) as single agents and in combination with Compound (1) against SCID female mice bearing human CRC CR-LRB-0011M patient-derived xenografts.

FIG. 3 graphically presents the antitumor activity of Compound (2) and Compound (3) as single agents and in combination with Compound (1) against SCID female mice bearing human CRC CR-LRB-0017P patient-derived xenografts.

FIG. 4 graphically presents the antitumor activity of Compound (2) and Compound (3) as single agents and in combination with Compound (1) against SCID female mice bearing human CRC CR-IGR-0023M patient-derived xenografts.

FIG. 5 graphically presents the antitumor activity of Compound (2) and Compound (3) as single agents and in combination with Compound (1) against SCID female mice bearing human CRC CR-LRB-0008M patient-derived xenografts.

FIG. 6 graphically presents the antitumor activity of Compound (2) and Compound (3) as single agents and in combination with Compound (1) against SCID female mice bearing human CRC CR-IGR-0032P patient-derived xenografts.

FIGS. 7A and 7B present plots of the mean (SD) plasma concentration of Compound (1) and Compound (2), respectively on day 15.

FIG. 8 presents a waterfall plot of 37 evaluable subjects from phase 1 trial.

FIG. 9 shows CT scans of a patient with low grade serous ovarian cancer, before and after two cycles of combination therapy with Compound (1) and Compound (2).

FIG. 10 provides a bar plot showing time on treatment and overall tumor response based on RECIST 1.1 for 53 evaluable subjects.

### DETAILED DESCRIPTION

In one aspect, methods for treating patients with cancer are provided. In one embodiment, the methods comprise administering to the patient a therapeutically effective amount of a MEK inhibitor and a therapeutically effective amount of a PI3K inhibitor, as further described below.

In one embodiment, the inventive methods and compositions comprise a MEK inhibitor having the following structural formula:

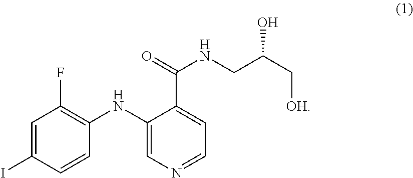 [SMILES: O=C(NC[C@H](O)CO)c1ccncc1Nc1ccc(I)cc1F]

The MEK inhibitor according to formula (1), N—((S)-2,3-dihydroxypropyl)-3-(2-fluoro-4-iodo-phenylamino)isonicotinamide, is referred to herein as “Compound (1)”. The preparation, properties, and MEK-inhibiting abilities of Compound (1) are provided in, for example, International Patent Publication No. WO 06/045514, particularly Example 115 and Table 1 therein. The entire contents of WO 06/045514 are incorporated herein by reference. Neutral and salt forms of the compound of Formula (1) are all considered herein.

In other embodiments, the inventive methods and compositions comprise a PI3K inhibitor having the following structure:

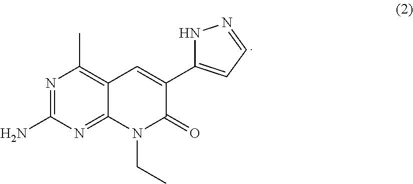 [SMILES: CCn1c(=O)c(-c2ccn[nH]2)cc2c(C)nc(N)nc21]

The PI3K inhibitor according to formula (2), 2-amino-8-ethyl-4-methyl-6-(1H-pyrazol-5-yl)pyrido[2,3-d]pyrimidin-7(8H)-one, is referred to herein as “Compound (2)”. The preparation and properties of Compound (2) are provided in, for example, International Patent Publication No. WO 07/044813, particularly Example 56 therein. The entire contents of WO 07/044813 and International Application No. PCT/US2011/063871 are incorporated herein by reference.

In other embodiments, the inventive methods and compositions comprise a PI3K inhibitor having the following structure:

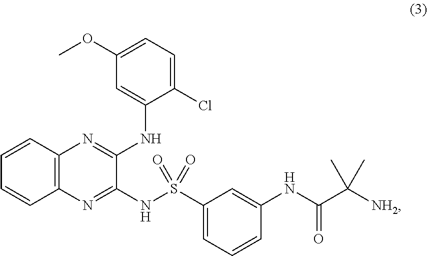 [SMILES: COc1ccc(Cl)c(Nc2nc3ccccc3nc2NS(=O)(=O)c2cccc(NC(=O)C(C)(C)N)c2)c1]

or tautomer thereof.

The PI3K inhibitor according to formula (3), N—(3-{[(3-{[2-chloro-5-(methoxy)phenyl]amino}quinoxalin-2-yl)amino]sulfonyl}phenyl)-2-methylalaninamide, or tautomer thereof, is referred to herein as “Compound (3)”. The preparation and properties of Compound (3) are provided in, for example, International Patent Publication No. WO 07/044729, particularly Example 357 therein. The entire contents of WO 07/044729 are incorporated herein by reference.

In some embodiments, the compounds described above are unsolvated. In other embodiments, one or both of the compounds used in the method are in solvated form. As known in the art, the solvate can be any of pharmaceutically acceptable solvent, such as water, ethanol, and the like. In general, the presence of a solvate or lack thereof does not have a substantial effect on the efficacy of the MEK or PI3K inhibitor described above.

Although the compounds in Formula (1) and Formula (2) are depicted in their neutral forms, in some embodiments, these compounds are used in a pharmaceutically acceptable salt form. The salt can be obtained by any of the methods well known in the art, such as any of the methods and salt forms elaborated upon in WO 07/044729, as incorporated by reference herein. A “pharmaceutically acceptable salt” of the compound refers to a salt that is pharmaceutically acceptable and that retains pharmacological activity. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, or S. M. Berge, et al., “Pharmaceutical Salts,” J. Pharm. Sci., 1977;66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, as well as those salts formed with organic acids, such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid.

In a first set of embodiments, the MEK inhibitor of formula (1) is administered simultaneously with the PI3K inhibitor of formula (2). Simultaneous administration typically means that both compounds enter the patient at precisely the same time. However, simultaneous administration also includes the possibility that the MEK inhibitor and PI3K inhibitor enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds.

In one example, wherein the compounds are in solution, simultaneous administration can be achieved by administering a solution containing the combination of compounds. In another example, simultaneous administration of separate solutions, one of which contains the MEK inhibitor and the other of which contains the PI3K inhibitor, can be employed. In one example wherein the compounds are in solid form, simultaneous administration can be achieved by administering a composition containing the combination of compounds.

In other embodiments, the MEK and PI3K inhibitors are not simultaneously administered. In this regard, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient. In one set of embodiments, the MEK inhibitor is administered before the PI3K inhibitor. In another set of embodiments, the PI3K inhibitor is administered before the MEK inhibitor. The time difference in non-simultaneous administrations is typically greater than 1 minute, and can be, for example, precisely, at least, up to, or less than 5 minutes, 10 minutes, 15 minutes, 30 minutes, 45 minutes, 60 minutes, two hours, three hours, six hours, nine hours, 12 hours, 24 hours, 36 hours, or 48 hours.

In one set of embodiments, one or both of the MEK and PI3K inhibitors are administered in a therapeutically effective (i.e., therapeutic) amount or dosage. A “therapeutically effective amount” is an amount of the MEK or PI3K inhibitor that, when administered to a patient by itself, effectively treats the cancer (for example, inhibits tumor growth, stops tumor growth, or causes tumor regression). An amount that proves “therapeutically effective amount” in a given instance, for a particular subject, may not be effective for 100% of subjects similarly treated for the disease or condition under consideration, even though such dosage is deemed a “therapeutically effective amount” by skilled practitioners. The amount of the compound that corresponds to a therapeutically effective amount is strongly dependent on the type of cancer, stage of the cancer, the age of the patient being treated, and other facts. In general, therapeutically effective amounts of these compounds are well-known in the art, such as provided in the supporting references cited above.

In another set of embodiments, one or both of the MEK and PI3K inhibitors are administered in a sub-therapeutically effective amount or dosage. A sub-therapeutically effective amount is an amount of the MEK or PI3K inhibitor that, when administered to a patient by itself, does not completely inhibit over time the biological activity of the intended target.

In some embodiments, the effective amount produces at least one therapeutic effect selected from the group consisting of reduction in size of a lung tumor, reduction in metastasis, complete remission, partial remission, stable disease, increase in overall response rate, or a pathologic complete response.

Whether administered in therapeutic or sub-therapeutic amounts, the combination of MEK inhibitor and PI3K inhibitor should be effective in treating the cancer. A sub-therapeutic amount of MEK inhibitor can be an effective amount if, when combined with the PI3K inhibitor, the combination is effective in the treatment of a cancer.

In some embodiments, the combination of compounds exhibits a synergistic effect (i.e., greater than additive effect) in treating the cancer, particularly in reducing a tumor volume in the patient. In different embodiments, depending on the combination and the effective amounts used, the combination of compounds can either inhibit tumor growth, achieve tumor stasis, or even achieve substantial or complete tumor regression.

In some embodiments, Compound (1) is administered at a dosage of about 7-120 mg po qd. Compound (2) can be administered at a dosage of about 15-90 mg po qd. In one embodiment, combination treatment comprises at least one cycle, wherein the cycle is a period of 3 weeks, wherein for each cycle the Compound (2) or pharmaceutically acceptable salt thereof is administered at a daily dose of about 30, 50, 70 or 90 mg and Compound (1) or pharmaceutically acceptable salt thereof is administered at a daily dose of about 15, 30, 60 or 90 mg.

As used herein, the term “about” generally indicates a possible variation of no more than 10%, 5%, or 1% of a value. For example, “about 25 mg/kg” will generally indicate, in its broadest sense, a value of 22.5-27.5 mg/kg, i.e., 25±10 mg/kg.

While the amounts of MEK and PI3K inhibitors should result in the effective treatment of a cancer, the amounts, when combined, are preferably not excessively toxic to the patient (i.e., the amounts are preferably within toxicity limits as established by medical guidelines). In some embodiments, either to prevent excessive toxicity and/or provide a more efficacious treatment of the cancer, a limitation on the total administered dosage is provided. Typically, the amounts considered herein are per day; however, half-day and two-day or three-day cycles also are considered herein.

Different dosage regimens may be used to treat the cancer. In some embodiments, a daily dosage, such as any of the exemplary dosages described above, is administered once, twice, three times, or four times a day for three, four, five, six, seven, eight, nine, ten days or more, e.g. 21 days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day. In some embodiments, each dosage contains both the MEK and PI3K inhibitors, while in other embodiments, each dosage contains either the MEK or PI3K inhibitors. In yet other embodiments, some of the dosages contain both the MEK and PI3K inhibitors, while other dosages contain only the MEK or the PI3K inhibitor.

In some embodiments, the claimed combination treatment can be used to treat patients with a cancer selected from the group consisting of non-small cell lung cancer, breast cancer, pancreatic cancer, liver cancer, prostate cancer, bladder cancer, cervical cancer, thyroid cancer, colorectal cancer, liver cancer, and muscle cancer. In other embodiments, the cancer is selected from colorectal cancer, endometrial cancer, hematology cancer, thryoid cancer, triple negative breast cancer or melanoma. In another embodiment, the claimed combination treatment can be used to treat patients with one or more to the following cancers: pancreatic, thyroid, colorectal, non-small cell lung, endometrial, renal, breast, ovarian carcinoma and melanoma. In another embodiment the cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

The term “treating” or “treatment”, as used herein, indicates that the method has, at the least, mitigated abnormal cellular proliferation. For example, the method can reduce the rate of tumor growth in a patient, or prevent the continued growth of a tumor, or even reduce the size of a tumor.

In another aspect, methods for preventing cancer in a human are provided. In this regard, prevention denotes causing the clinical symptoms of the disease not to develop in a human that may be exposed to or predisposed to the disease but does not yet experience or display symptoms of the disease. The methods comprise administering to the patient a MEK inhibitor and a PI3K inhibitor, as described herein. In one example, a method of preventing cancer in a patient comprises administering to the patient a compound of Formula (1), or a pharmaceutically acceptable salt thereof, in combination with a compound of Formula (2), or a pharmaceutically acceptable salt thereof.

The MEK and PI3K inhibiting compounds, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A particular route of administration is oral, particularly one in which a convenient daily dosage regimen can be adjusted according to the degree of severity of the disease to be treated.

In another aspect, the instant application is directed to a composition that includes the MEK inhibitor shown in Formula (1) and a PI3K inhibitor shown in Formula (2). In some embodiments, the composition includes only the MEK and PI3K inhibitors described above. In other embodiments, the composition is in the form of a solid (e.g., a powder or tablet) including the MEK and PI3K inhibitors in solid form, and optionally, one or more auxiliary (e.g., adjuvant) or pharmaceutically active compounds in solid form. In other embodiments, the composition further includes any one or combination of pharmaceutically acceptable carriers (i.e., vehicles or excipients) known in the art, thereby providing a liquid dosage form.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Dosage forms suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms also may comprise buffering agents.

Solid dosage forms as described above can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a MEK or PI3K inhibitor compound described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds described herein with, for example, suitable non- irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration may include, for example, ointments, powders, sprays, and inhalants. The active component is admixed under sterile conditions with a physiologically acceptable carrier and any preservatives, buffers, or propellants as can be required. Ophthalmic formulations, eye ointments, powders, and solutions also can be employed.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compounds described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

In some embodiments, the composition does not include one or more other anti-cancer compounds. In other embodiments, the composition includes one or more other anti-cancer compounds. For example, administered compositions can comprise standard of care agents for the type of tumors selected for treatment.

In another aspect, kits are provided. Kits according to the invention include package(s) comprising compounds or compositions of the invention. In one embodiment, kits comprise Compound (1), or a pharmaceutically acceptable salt thereof, and Compound (2), or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit also can contain items that are not contained within the package but are attached to the outside of the package, for example, pipettes.

Kits can contain instructions for administering compounds or compositions of the invention to a patient. Kits also can comprise instructions for approved uses of compounds herein by regulatory agencies, such as the United States Food and Drug Administration. Kits also can contain labeling or product inserts for the inventive compounds. The package(s) and/or any product insert(s) may themselves be approved by regulatory agencies. The kits can include compounds in the solid phase or in a liquid phase (such as buffers provided) in a package. The kits also can include buffers for preparing solutions for conducting the methods, and pipettes for transferring liquids from one container to another.

Examples have been set forth below for the purpose of illustration and to describe certain specific embodiments of the invention. However, the scope of the claims is not to be in any way limited by the examples set forth herein.

### EXAMPLES

### Example 1 Preclinical Experiments

Six patient-derived colorectal cancer (CRC) xenograft models were utilized to explore the antitumor activity and the terminal pharmacodynamic (PD) impact of Compound (2) and Compound (3) as single agents and in combination with the MEK inhibitor, Compound (1). The models chosen harbored either, single KRAS mutations, dual KRAS and BRAF mutations or dual KRAS and PIK3CA mutations (Table 1).

TABLE 1
Patient-derived CRC xenograft models selected for activity studies with
Compound (2) or Compound (3) in combination with Compound (1).

| | CRC Xenograft | Mutation Status | | |
|---|---|---|---|---|
| | Models | KRAS | PIK3CA | BRAF |
| 1. | CR-IC0013-M | G13D | Wt | V600E |
| 2. | CR-LRB-0011-M | G12V | Wt | wt |
| 3. | CR-LRB-0017-P | G12D | Wt | wt |
| 4. | CR-IGR-0023-M | G12D | E542K | wt |
| 5. | CR-LRB-0008-M | G12V | E545K | wt |
| 6. | CR-IGR-0032-P | G12D | E545K | wt |

The standard experimental design for these studies involved PO dosing of Compound (2) (20 mg/kg qd), Compound (3) (75 mg/kg qd) and Compound (1) (20 mg/kg qd, except in 1 model, CR-IGR-0032-P where a dose of 10 mg/kg qd was used). In the combination groups a single agent dose of Compound (2) or Compound (3) was combined with a dose of Compound (1). Dosing was initiated once the established solid tumors were staged, approximately 150 to 170 mm³ staging size for most subcutaneous xenograft models. Typically, dosing groups were comprised of 7 or 8 animals per dose level. Throughout the dosing period, tumor size was measured at least twice weekly and group body weights were recorded daily. The terminal PD impact was evaluated on phospho-proteins of the MAPK and PI3K/AKT pathways in extracts from tumors collected 4 hours after the last treatment.

### Results

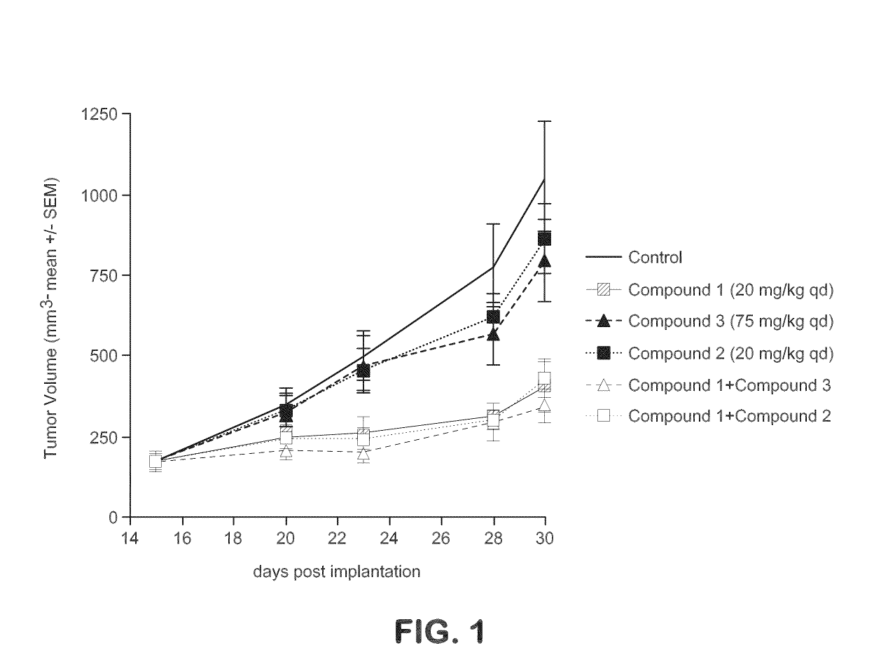

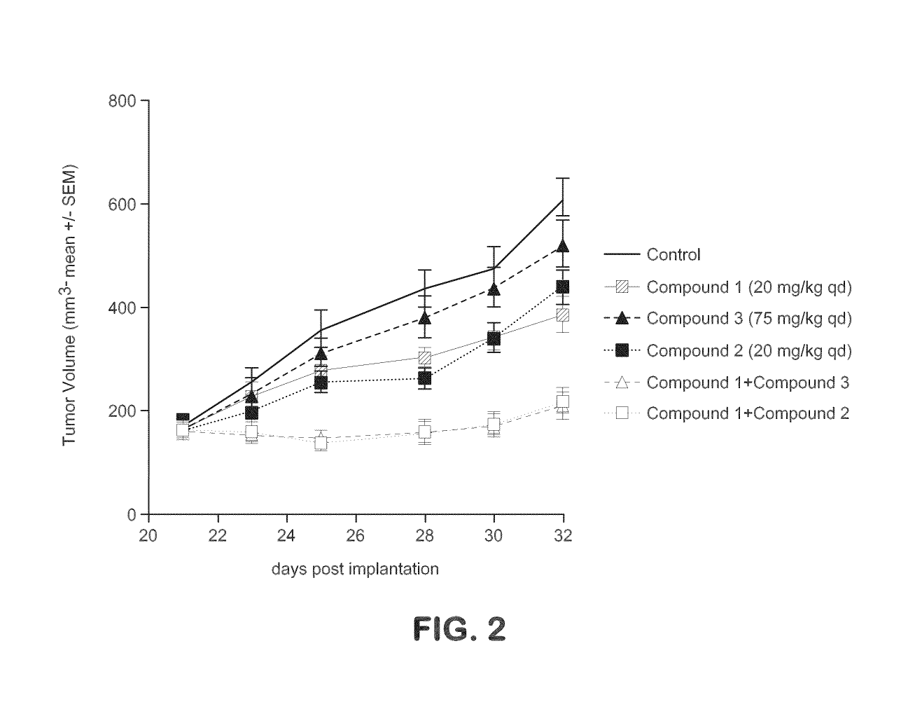

The results are summarized in Table 2-8 and FIGS. 1- 6.

In CR-IC-0013-M (dual G13D KRAS and V600E BRAOF mutant), Compound (1) showed antitumor activity as single agent, and this activity was related to a strong inhibition of pMAPK pathway. Compound (2) and Compound (3) led to potent inhibition of the PI3K pathway, but no impact on the MAPK pathway. Both combinations displayed anti-tumor activity and potent inhibition of biomarkers of the MAPK and PI3K pathways.

In CR-LRB-0011-M (G12V KRAS mutant), combinations of Compound (1) with Compound (2) or Compound (3) displayed potent antitumor activity. Compound (1) displayed potent inhibition of p-ERK and some inhibition of the mTORC 1 pathway (pS240/244 S6RP and pT37/46 4E-BP1). Both PI3K inhibitors as single agents alone, displayed inhibition of the mTORC1 pathway.

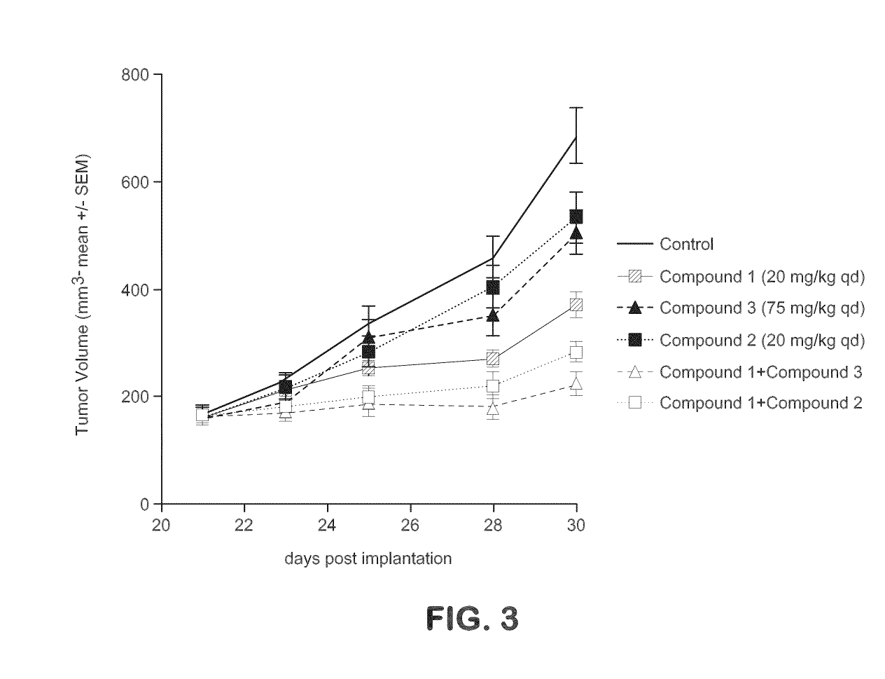

In CR-LRB-0017-P (G12D KRAS mutant), potent inhibition of p-ERK by Compound (1) was observed leading to modest anti-tumor activity as single agent. Combination of Compound (1) with Compound (2) or Compound (3) produced an additive effect on tumor growth activity and the mTORC1 pathway (pS240/244 S6RP and pT37/46 4E-BP1) (Tables 2 & 5; FIG. 3).

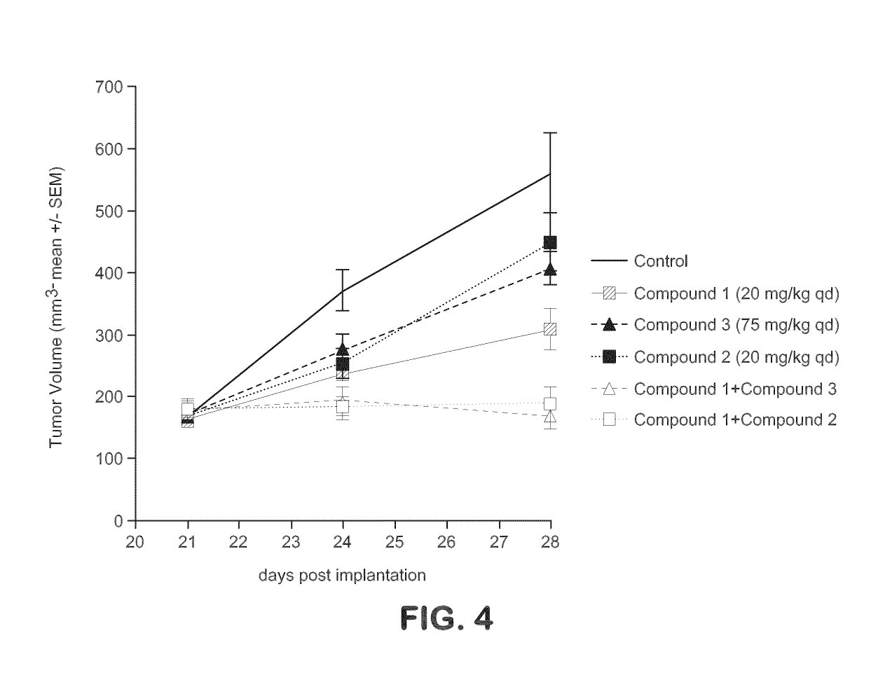

In CR-IGR-0023-M (dual G12D KRAS and E542K PIK3CA mutant), potent inhibition of p-ERK by Compound (1) was observed. Compound (2) and Compound (3) displayed inhibition of the mTORC 1 and mTORC2 pathway. The combination of Compound (1) with Compound (2) or Compound (3) displayed potent anti-tumor activity and additive effects in both the mTORC2 (pS473Akt and pT246 PRAS40) and mTORC1 pathways (pS240/244 S6RP and pT37/46 4E-BP1) (Tables 2 & 6; FIG. 4).

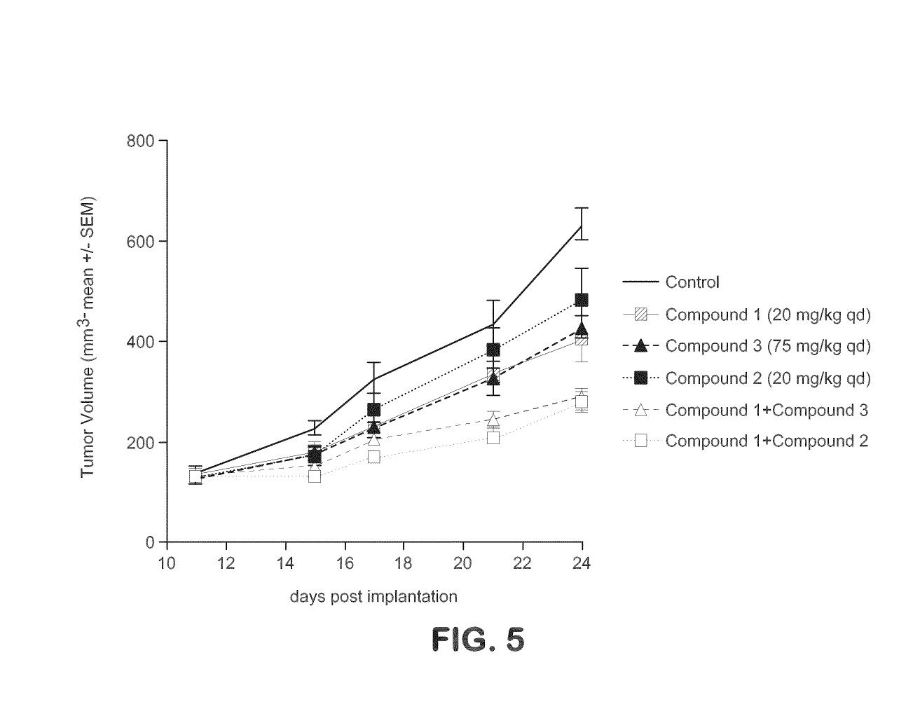

In CR-LRB-0008-M (dual G12V KRAS and E545K PIK3CA mutant), potent inhibition of p-ERK by Compound (1) was observed. Both Compound (2) and Compound (3) as single agents displayed inhibition of the mTORC1 and mTORC2 pathway. The combination of Compound (1) with Compound (2) or Compound (3) displayed potent anti-tumor activity additive effects on the mTORC2 (pS473Akt and pT246 PRAS40) and mTORC1 pathways (pS240/244 S6RP and pT37/46 4E-BP1) (Tables 2 & 7; FIG. 5).

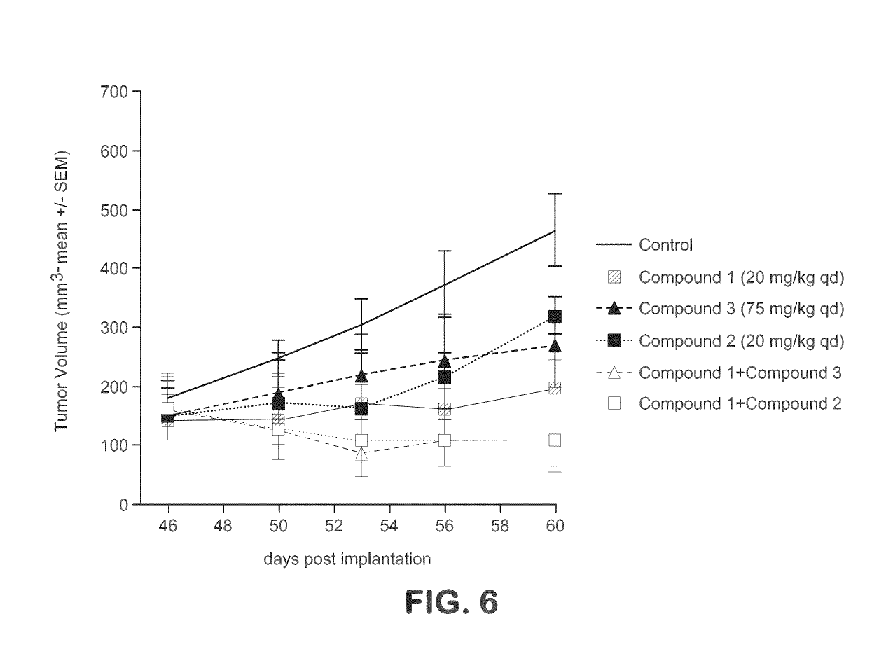

In CR-IGR-0032-P (dual G12D KRAS and E545K PIK3CA mutant), potent inhibition of p-ERK by Compound (1) (at 10 mg /kg qd) was observed correlating with anti-tumor activity as single agent. Compounds (2) and (3) displayed an inhibition on the mTORC1 and mTORC2 pathways. The combination of Compound (1) with Compound (2) or Compound (3) displayed potent anti-tumor activity and additive effects on the mTORC2 (pS473Akt and pT246 PRAS40) and mTORC1 pathways (Tables 2 & 8; FIG. 6).

In summary, in the models tested, Compound (2) and Compound (3) in combination with Compound (1) were more active than the single agents alone, except in the dual KRAS and BRAF mutant primary CRC xenograft model (CR-IC-0013-M). The three CRC patient-derived xenograft models carrying both KRAS and PIK3CA mutations (CR-IGR-0023-M, CR-LRB-0008-M & CR-IGR-0032-P) showed similar activities in that the combination of either Compound (2) or Compound (3) with Compound (1) showed potent antitumor activity as compared to single agents alone, which correlated with additive effects on the inhibition of markers of the mTORC1 and mTORC2 pathways. In the two colorectal cancer patient-derived xenograft models carrying single KRAS mutations (CR-LRB-0011-M and CR-LRB-0017-P), both combinations displayed potent antitumor activity which was greater than that observed with the individual agents.

Regarding tolerability, a dosage producing a 15% body weight loss (bwl) during three consecutive days (mean of group), 20% bwl during one day or 10% or more drug deaths, was considered an excessively toxic dosage, unless cachexia leading to bwl was observed in the control untreated groups. Compound (3), Compound (2) and Compound (1) either as single agents or in combinations were tolerated in the majority of the CRC patient-derived xenograft models as determined by no significant body weight loss during the course of the study.

TABLE 2
Activity of Compounds (1), (2) and (3) as single agents and in combination
in CRC xenograft models.

| | | | | Compound (1) | Compound (1) |
|---|---|---|---|---|---|
| | | | | (20 mg/kg qd) + | (20 mg/kg qd) + |
| | | | | Compound | Compound |
| | Compound (2) | Compound (3) | Compound (1) | (2) (20 | (3) (75 mg/kg |
| ΔT/ΔC values | (20 mg/kg qd) | (75 mg/kg qd) | (20 mg/kg qd) | mg/kg qd) | qd) |
| CR-IC-0013-M | 64% | 70% | 22% | 28% | 14% |
| (KRAS/BRAE) | (p = 0.9999) | (p = 0.9361) | (p = 0.0003) | (p = 0.0055) | (p < 0.0001) |
| Day 30 | | | | | |
| CR-LRB-0011-M | 71% | 87% | 51% | 12% | 8% |
| (KRAS) | (p = 0.1927) | (p = 0.6687) | (p = 0.0053) | (p < 0.0001) | (p < 0.0001) |
| Day 32 | | | | | |
| CR-LRB-0017-P | 61% | 65% | 35% | 21% | 8% |
| (KRAS) | (p = 0.8816) | (p = 0.8136) | (p = 0.0146) | (p < 0.0001) | (p < 0.0001) |
| Day 30 | | | | | |
| CR-IGR-0023-M | 75% | 70% | 44% | 0% | 0% |
| (KRAS/PIK3CA) | (p < 0.0136) | (p = 0.0314) | (p < 0.0001) | (p < 0.0001) | (p < 0.0001) |
| Day 28 | | | | | |
| CR-LRB-008-M | 65% | 60% | 52% | 28% | 28% |
| (KRAS/PIK3CA) | (p = 0.0189) | (p = 0.0394) | (p = 0.0064) | (p < 0.0001) | (p < 0.0001) |
| Day 24 | | | | | |
| CR-IGR-0032-P | 55% | 34% | 17%* | <0%* | <0%* |
| (KRAS/PIK3CA) | (p = 0.3547) | (p = 0.0209) | (p = 0.0006) | (p < 0.0001) | (p < 0.0001) |
| Day 60 | | | | | |

*In the CR-IGR-0032-P mode, Compound (1) was used at a dose of 10 mg/kg qd

TABLE 3
Summary of terminal PD impact of Compound (2) and Compound (3)
as single agents and in combination with Compound (1) against human CRC
CR-IC-0013-M patient-derived xenografts on biomarkers of the PI3K & MAPK pathways.

| | Change versus Vehicle | | | | | |
|---|---|---|---|---|---|---|
| | pT202/Y204 | pS217/ | | pT246 | pS240/ | pT37/ |
| Group | ERK | 221 MEK | pS473AKT | PRAS40 | 244 S6RP | 464E-BP1 |
| Compound (1) | −84 | 64 | 8 | −7 | −7 | −22 |
| 20 mg/kg | | | | | | |
| Compound (2) | −23 | −14 | −71 | −55 | −42 | −54 |
| 20 mg/kg | | | | | | |
| Compound (3) | 15 | 14 | −60 | −46 | −22 | −40 |
| 75 mg/kg | | | | | | |
| Compound (2) | −87 | 88 | −66 | −56 | −53 | −56 |
| 20 mg/kg + | | | | | | |
| Compound (1) | | | | | | |
| 20 mg/kg | | | | | | |
| Compound (3) | −89 | 107 | −53 | −49 | −51 | −44 |
| 75 mg/kg + | | | | | | |
| Compound (1) | | | | | | |
| 20 mg/kg | | | | | | |

The change of protein biomarker level was calculated as % change = [(mean AU of treated group - mean AU of control group)/mean AU of control group]*100

TABLE 4
Summary of terminal PD impact of Compound (2) and Compound (3)
as single agents and in combination with Compound (1) against human CRC
CR-LRB-0011-M patient-derived xenografts on biomarkers of the PI3K & MAPK pathways.

| | Change versus Vehicle | | | | | |
|---|---|---|---|---|---|---|
| | pT202/Y204 | pS217/ | | pT246 | pS240/ | pT37/ |
| Group | ERK | 221 MEK | pS473AKT | PRAS40 | 244 S6RP | 464E-BP1 |
| Compound (1) | −78 | 83 | −23 | 4 | −48 | −31 |
| 20 mg/kg | | | | | | |
| Compound (2) | −1 | 9 | −11 | 10 | −52 | −24 |
| 20 mg/kg | | | | | | |
| Compound (3) | −1 | 15 | −30 | 4 | −23 | −7 |
| 75 mg/kg | | | | | | |
| Compound (2) | −80 | 124 | −14 | −12 | −67 | −29 |
| 20 mg/kg + | | | | | | |
| Compound (1) | | | | | | |
| 20 mg/kg | | | | | | |
| Compound (3) | −74 | 169 | −14 | −6 | −47 | −36 |
| 75 mg/kg + | | | | | | |
| Compound (1) | | | | | | |
| 20 mg/kg | | | | | | |

The change of protein biomarker level was calculated as % change = [(mean AU of treated group - mean AU of control group)/mean AU of control group]*100

TABLE 5
Summary of terminal PD impact of Compound (2) and Compound (3) as single
agents and in combination with Compound (1) against human CRC CR-LRB-0017-P
patient-derived xenografts on biomarkers of the PI3K, MAPK and apoptosis pathways.

| | Change versus Vehicle | | | | | |
|---|---|---|---|---|---|---|
| | pT202/Y204 | pS217/ | | pT246 | pS240/ | pT37/ |
| Group | ERK | 221 MEK | pS473AKT | PRAS40 | 244 S6RP | 464E-BP1 |
| Compound (1) | −70 | 32 | 84 | 17 | −41 | −14 |
| 20 mg/kg | | | | | | |
| Compound (2) | 6 | −4 | 118 | 42 | −21 | −3 |
| 20 mg/kg | | | | | | |
| Compound (3) | 19 | 9 | 9 | −5 | 5 | −16 |
| 75 mg/kg | | | | | | |
| Compound (2) | −85 | 52 | 47 | −14 | −59 | −35 |
| 20 mg/kg + | | | | | | |
| Compound (1) | | | | | | |
| 20 mg/kg | | | | | | |
| Compound (3) | −73 | 203 | 14 | −25 | −48 | −39 |
| 75 mg/kg + | | | | | | |
| Compound (1) | | | | | | |
| 20 mg/kg | | | | | | |

The change of protein biomarker level was calculated as % change = [(mean AU of treated group - mean AU of control group)/mean AU of control group]*100

TABLE 6
Summary of terminal PD impact of Compound (2)
and Compund (3) as single agents and in combination with
Compound (1) against human CRC CR-LRB-0023-M patient-
derived xenografts on biomarkers of the PI3K, MAPK and
apoptosis pathways.

| | Change versus Vehicle | | | | | |
|---|---|---|---|---|---|---|
| | pT202/ | pS217/ | | | pS240/ | pT37/ |
| | Y204 | 221 | | pT246 | 244 | 464E- |
| Group | ERK | MEK | pS473AKT | PRAS40 | S6RP | BP1 |
| Compound (1) | −78 | 219 | −16 | 15 | −24 | −32 |
| 20 mg/kg | | | | | | |
| Compound (2) | 22 | 25 | −24 | −16 | −41 | −29 |
| 20 mg/kg | | | | | | |
| Compound (3) | 26 | 34 | −52 | −12 | −8 | −23 |
| 75 mg/kg | | | | | | |
| Compound (2) | −83 | 497 | −68 | −52 | −88 | −64 |
| 20 mg/kg + | | | | | | |
| Compound (1) | | | | | | |
| 20 mg/kg | | | | | | |
| Compound (3) | −70 | 477 | −53 | −40 | −68 | −44 |
| 75 mg/kg + | | | | | | |
| Compound (1) | | | | | | |
| 20 mg/kg | | | | | | |

The change of protein biomarker level was calculated as % change = [(mean AU of treated group - mean AU of control group)/mean AU of control group]*100

TABLE 7
Summary of terminal PD impact of Compound (2) and Compound (3) as single
agents and in combination with Compound (1) against human CRC CR-LRB-0008-M
patient-derived xenografts on biomarkers of the PI3K, MAPK and apoptosis pathways.

| | Change versus Vehicle | | | | | |
|---|---|---|---|---|---|---|
| | pT202/ | pS217/ | | | pS240/ | |
| | Y204 | 221 | | pT246 | 244 | pT37/ |
| Group | ERK | MEK | pS473AKT | PRAS40 | S6RP | 464E−BP1 |
| Compound (1) | −57 | 235 | −12 | −1 | −33 | −23 |
| 20 mg/kg | | | | | | |
| Compound (2) | 50 | 29 | −64 | −18 | −68 | −42 |
| 20 mg/kg | | | | | | |
| Compound (3) | 14 | 23 | −43 | −6 | −37 | −4 |
| 75 mg/kg | | | | | | |
| Compound (2) | −72 | 277 | −83 | −53 | −73 | −62 |
| 20 mg/kg + | | | | | | |
| Compound (1) | | | | | | |
| 20 mg/kg | | | | | | |
| Compound (3) | −70 | 384 | −66 | −28 | −66 | −38 |
| 75 mg/kg + | | | | | | |
| Compound (1) | | | | | | |
| 20 mg/kg | | | | | | |

The change of protein biomarker level was calculated as % change = [(mean AU of treated group - mean AU of control group)/mean AU of control group]*100

TABLE 8
Summary of terminal PD impact of Compound (2) and Compound (3) as single
agents and in combination with Compound (1) against human CRC CR-IGR-0032-P
patient-derived xenografts on biomarkers of the PI3K, MAPK and apoptosis pathways.

| | Change versus Vehicle | | | | | |
|---|---|---|---|---|---|---|
| | pT202/ | | | | pS240/ | |
| | Y204 | | | pT246 | 244 | pT37/46 |
| Group | ERK | pT308AKT | pS473AKT | PRAS40 | S6RP | 4E−BP1 |
| Compound (1) | −79 | −18 | −22 | −8 | −40 | −19 |
| 10 mg/kg | | | | | | |
| Compound (2) | 38 | −69 | −67 | −34 | −64 | −40 |
| 20 mg/kg | | | | | | |
| Compound (3) | −8 | −68 | −58 | −20 | −36 | −16 |
| 75 mg/kg | | | | | | |
| Compound (2) | −71 | −79 | −78 | −47 | −69 | −58 |
| 20 mg/kg + | | | | | | |
| Compound (1) | | | | | | |
| 10 mg/kg | | | | | | |
| Compound (3) | −70 | −80 | −74 | −51 | −66 | −45 |
| 75 mg/kg + | | | | | | |
| Compound (1) | | | | | | |
| 10 mg/kg | | | | | | |

The change of protein biomarker level was calculated as % change = [(mean AU of treated group - mean AU of control group)/mean AU of control group]*100

### Example 2 Phase 1b Trial in Patients Having Solid Tumors

A non-comparative, open-label, nonrandomized, Phase Ib, combination, dose escalation trial, is conducted using a classical “3+3” design in dose escalation cohorts. In parallel to dose escalation cohorts, additional subjects may be enrolled in lower dose level (LDL) cohorts as per decision of the safety monitoring committee (SMC) in order to further evaluate safety, PK, anti-tumor and Pd activity. After the maximum tolerated dose (MTD) is reached, MTD cohort(s) will be expanded with additional subjects to confirm the MTD(s). After confirmation of the MTD(s), additional subjects with specific tumor diagnosis will be enrolled as per SMC decision in up to four disease specific expansion cohort.

A maximum of 90 subjects are expected to be enrolled and treated in the dose escalation and LDL/MTD cohorts of the trial. Approximately an additional 80 subjects are planned to be enrolled in four disease specific expansion cohorts of the trial in order to have 18 evaluable subjects per disease specific cohort.

### Objectives

The primary objective is to determine the Maximum Tolerated Dose(s) (MTD[s]) of Compound (1) and Compound (2) combination therapy administered orally to adult subjects with locally advanced or metastatic solid tumors.

Secondary objectives include the following:

To characterize the safety and tolerability of Compound (1) and Compound (2) combination therapy administered orally to adult subjects with locally advanced or metastatic solid tumors.

To evaluate the pharmacokinetic (PK) profile of Compound (1) and Compound (2) combination therapy.

To evaluate the pharmacodynamic (Pd) effect of Compound (1) and Compound (2) combination therapy.

To explore potential correlations between alterations of PI3K/PTEN pathway and MAPK pathway components/modulators, oncogenes/tumor suppressor genes that directly and/or indirectly are involved in these pathways and the response following Compound (1) and Compound (2) combination therapy.

To describe preliminary anti-tumor activity, based on response rate (RR), and disease control rate (DCR) in subjects with evaluable disease who received Compound (1) and Compound (2) combination therapy.

### Inclusion Criteria

1. The subject with advanced solid tumors for which there is no approved therapy has any advanced solid tumor with diagnosed alteration in one or more of the following genes (PTEN, BRAF, KRAS, NRAS, PI3KCA, ErbBl, ErbB2, MET, RET, c-KIT GNAQ, GNA11) and/or has a histologically or cytologically confirmed diagnosis of one of the following solid tumors: pancreatic, thyroid, colorectal, non-small cell lung, endometrial, renal, breast, ovarian carcinoma and melanoma. Based on the SMC decision, enrollment in the MTD expansion cohorts may be further limited to the indication(s) in which strong signals of activity are present if such is/are identified during the dose escalation. In addition, subjects enrolled in disease specific expansion cohorts must have specific tumor diagnosis as specified below.

2. The subject has archived tumor tissue available for transfer to the Sponsor.

3. The subject enrolled at LDL cohorts and MTD expansion cohorts must also have tumor accessible for biopsies and agree to pretreatment and ontreatment tumor biopsies. For the subjects enrolled in disease specific expansion cohorts, the tumor accessibility for biopsy is not mandatory and the pretreatment and on-treatment tumor biopsies are optional.

4. The subject has measurable or evaluable disease by Response Evaluation Criteria In Solid Tumors (RECIST) v1.1.

5. The subject is aged ≧18 years.

6. The subject has read and understood the Informed Consent Form (ICF) and is willing and able to give informed consent, fully understands requirements of the trial and is willing to comply with all trial visits and assessments. Consent must be given before any trial related activities.

7. The subject has performance status score of ≦1 according to the Eastern Cooperative Oncology Group (ECOG) scale.

8. Female subjects of childbearing potential must have a negative blood pregnancy test at the screening visit. For the purposes of this trial, women of childbearing potential are defined as: “All female subjects after puberty unless they are post-menopausal for at least two years, are surgically sterile or are sexually inactive”.

9. Female subjects of childbearing potential and male subjects with female partners of childbearing potential must be willing to avoid pregnancy by using an adequate method of contraception for 2 weeks prior to screening, during and at least four weeks after the last dose of trial medication. Adequate contraception is defined as follows: two-barrier method or one barrier method with a spermicide or intrauterine device. The use of hormonal contraceptives should be avoided due to a possible drug-drug interaction.

### Exclusion Criteria

1. The subject has previously been treated with a PI3K inhibitor or a MEK inhibitor and taken off treatment due to treatment related adverse events. In the LDL and MTD expansion cohorts, all subjects who have been previously treated with a PI3K inhibitor or a MEK inhibitor will be excluded.

2. The subject has received: a. Chemotherapy, immunotherapy, hormonal therapy, biologic therapy, or any other anticancer therapy within 28 days of Day 1 of trial drug treatment (6 weeks for nitrosureas or mitomycin C); b. Any investigational agent within 28 days of Day 1 of trial drug treatment; c. Extensive prior radiotherapy on more than 30% of bone marrow reserves, or prior bone marrow/stem cell transplantation.

3. The subject has not recovered from toxicity due to prior therapy to baseline or Common Terminology Criteria for Adverse Events (CTCAE) of Grade 1 or less (except alopecia).

4. The subject has poor organ and marrow function as defined by the following:
a. absolute neutrophil count ≦1500/mm3
b. platelets ≦100,000/mm3
c. hemoglobin ≦9 g/dL
d. bilirubin ≧1.5×the upper limit of normal (ULN)
e. alanine aminotransferase and aspartate aminotransferase ≧2.5×the ULN
f. serum creatinine ≧1.5×the ULN or measure creatinine clearance ≦60 mL/min (Cockroft-Gault formula)

5. The subject has history of central nervous system (CNS) metastases (unless subject has been previously treated for CNS metastases, is stable by computed tomography (CT) scan without evidence of cerebral edema, and has no requirements for corticosteroids or anti-convulsants for a minimum of 2 weeks prior to entry into the trial) OR the subject has a primary brain tumor.

6. The subject has history of difficulty swallowing, malabsorption or other chronic gastrointestinal disease or conditions that may hamper compliance and/or absorption of the tested product.

7. The subject has history of recent major surgery or trauma (within the last 28 days), unhealing/open wounds, diabetic ulcers, recent drainage of significant volumes of ascites or pleural effusion only if drainage can potentially lead to a hemodynamic instability.

8. The subject has history of congestive heart failure, unstable angina, a myocardial infarction, cardiac conduction abnormality or pacemaker or a stroke within 3 months prior to entering the trial.

9. The subject has a baseline corrected QT (QTc) interval on screening electrocardiogram (ECG) ≧460 ms or left ventricular ejection fraction (LVEF) <40% on screening assessment.

10. The subject has history of retinal degenerative disease (hereditary retinal degeneration or age-related macular degeneration), history of uveitis, history of retinal vein occlusion, or has medically relevant abnormalities identified on screening ophthalmologic examination.

11. The subject has history of uncontrolled intercurrent illness including but not limited to an active infection, hypertension, or uncontrolled diabetes (e.g. HgbAlc≧8%) that would limit compliance with trial requirements.

12. The subject is known to be positive for the human immunodeficiency virus, or has active hepatitis B, and C, or other chronic viral infections.

13. The subject has psychiatric illness/social situation(s) that would limit compliance with trial requirements.

14. The subject is pregnant or/and lactating.

15. The subject has participated in another clinical trial within the past 30 days.

16. The subject has history of other significant disease that in the Investigator's opinion would exclude the subject from the trial.

17. The subject has known hypersensitivity to the trial treatment(s).

18. The subject has legal incapacity or limited legal capacity.

### Inclusion and Exclusion Criteria for Disease Specific Expansion Cohorts

Subject enrolled in disease specific expansion cohorts must fulfill all the inclusion/exclusion criteria listed above with the following restrictions to the Inclusion Criterion 1:

Only subjects with one of the following histologically confirmed cancer diagnosis will be included:
Relapsed or refractory KRAS or NRAS mutated metastatic non small cell lung cancer (NSCLC) with no approved therapies OR
Relapsed or refractory metastatic triple negative breast cancer (TNBC, defined as estrogen progesterone and HER2 negative carcinoma of the breast with no approved therapies OR
Relapsed or refractory metastatic CRC with dual KRAS and PIK3CA mutation and with no approved therapies OR p1 BRAF V600E/K mutated unresectable or metastatic melanoma after progression on BRAF inhibitors.

For subjects enrolled in the disease specific expansion cohorts the results of ER, PR, HER2 as well as mutational status of KRAS, NRAS, BRAF and PIK3CA genes must be available as relevant to the diagnosis. If PIK3CA mutation is not assessed as part of primary tumor diagnosis it may be evaluated from the plasma (circulating DNA, see Section 7.6.6) during the screening period.

### Dose/Schedule

Both Compound (2) and Compound (1) will be taken together, in fasted state, continuously once daily (Q.D.). The starting dose of Compound (1) chosen for this combination is 15 mg Q.D. Compound (1) will be supplied as 4, 15 and 30 mg hard gelatin capsules. The starting dose of Compound (2) chosen for this combination trial is 30 mg Q.D. Compound (2) will be supplied as 10, 30, and 40 capsules.

The dose escalation scheme is presented in Table 9.

TABLE 9

| | Compound (1) | | | | |
|---|---|---|---|---|---|
| | Administered per day | | | | |
| | | 15 mg | 30 mg | 60 mg | 90 mg |
| Compound (2) | 30 mg | DL1 | DL2a | | |
| Administered | 50 mg | DL2b | DL3 | DL4a | |
| per day | 70 mg | | DL4b | DL5 | DL6a |
| | 90 mg | | | DL6b | DL7 |

Subjects will be treated in 21-day treatment cycles until disease progression, intolerable toxicity, Investigator's decision to discontinue treatment, or withdrawal of consent by the subject. The duration of the trial for an individual subject will include:
(1) Screening and baseline evaluation 28-day period.
(2) Drug-drug interaction (DDI) evaluation period, 4 days when PK and Pd sampling is performed for each compound administered separately to enable an intraindividual cross-over comparison when the two IMPs are administered in combination, in order to assess their possible interaction (for subjects enrolled in the first dose level and MTD expansion cohorts and also at any additional dose levels (DL) if recommended by the SMC). DDI evaluation may be also performed in disease specific expansion cohorts if recommended by the SMC.
(3) Treatment period of at least 21 days (one cycle of trial treatment)
(4) Follow-up period of 30 (±3) days after the last IMP administration.

### Endpoints

The primary endpoint of this trial is the dose-limiting toxicity (DLT). The number and the proportion of subjects with DLTs will be used as the primary measure for the MTD determination.

Secondary endpoints include:
Safety parameters: treatment-emergent adverse events (TEAEs) (graded according to the NCI Common Terminology Criteria for Adverse Events (CTCAE) v4.0), laboratory tests, physical examinations, vital signs, ECGs, echocardiogram/MUGA scan, ophthalmologic assessments, etc. The number and the proportions of subjects with TEAEs and abnormal findings regarding any other safety parameter will be tabulated and reviewed for potential significance and clinical relevance.
Plasma PK parameters of Compound (1) (Cmax, tmax, AUC0-24, AUC□, AUC0-□, t1/2, CL/f, CLss/f, Vz/f, Vss/f, Racc(AUC), Racc(Cmax)).
Plasma PK parameters of Compound (2) (Cmax, tmax, AUC0-24, AUC [symbol] , AUC0 t1/2, CL/f, CLss/f, Vz/f, Vss/f, Racc(AUC), Racc(Cmax)).
Values and changes over time in Pd marker in peripheral blood mononuclear cells (PBMCs) including whole blood flow cytometry assay for pERK(T202/Y204) and pS6(S240/S244).
   Values and changes over time in exploratory Pd markers in pre- and on-treatment tumor biopsies (tumor sample collection optional in dose escalation, but mandatory in the MTD and LDL expansion cohorts) including immunohistochemistry (IHC) assay for:

Pd markers of the MAPK pathway such as pERK(T202/Y204) and pMEK(S217/221);

Pd markers of PI3K pathways such as p4EBP1(T70), pPRAS40(T246) and pS6(S240/S244);

Mechanistic biomarkers such as markers of proliferation (e.g. Ki67, Cyclin D1 or pRB) and apoptosis (e.g. cleaved Caspase3 or BIM).
Presence or absence of genetic variation associated to the MAPK (e.g. KRAS, BRAF) and PI3K pathways (e.g. PI3KCA) in tumor tissue, and correlation with tumor response for exploratory predictive analysis.
Predictive markers in plasma (e.g. circulating DNA).
Changes in levels of targeted pathways related markers expression in pre/post treatment plucked hair follicles.
Genetic variations in genes that may be involved in the adsorption, distribution, metabolism, and excretion (ADME), associated with differences in PK profile of Compound (1) in combination with Compound (2) (optional).
Response rate (defined as the proportion of evaluable subjects achieving a complete response [CR] or partial response [PR]) and DCR (defined as the proportion of evaluable subjects achieving CR, or PR, or stable disease [SD] ≧12 weeks) based on the Investigator tumor evaluations performed every 6 weeks in accordance with RECIST v1.1.

### Results

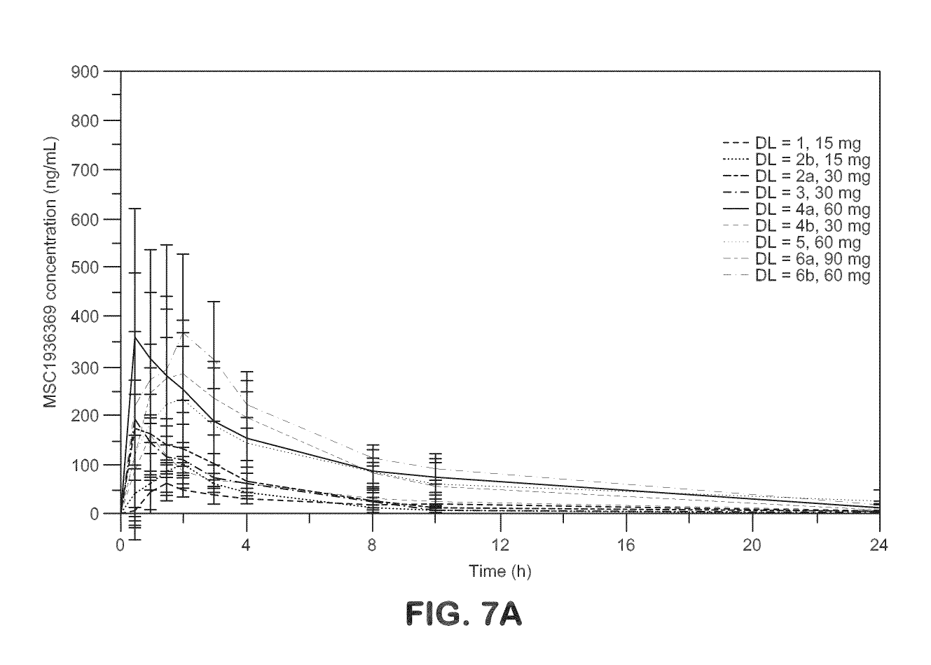

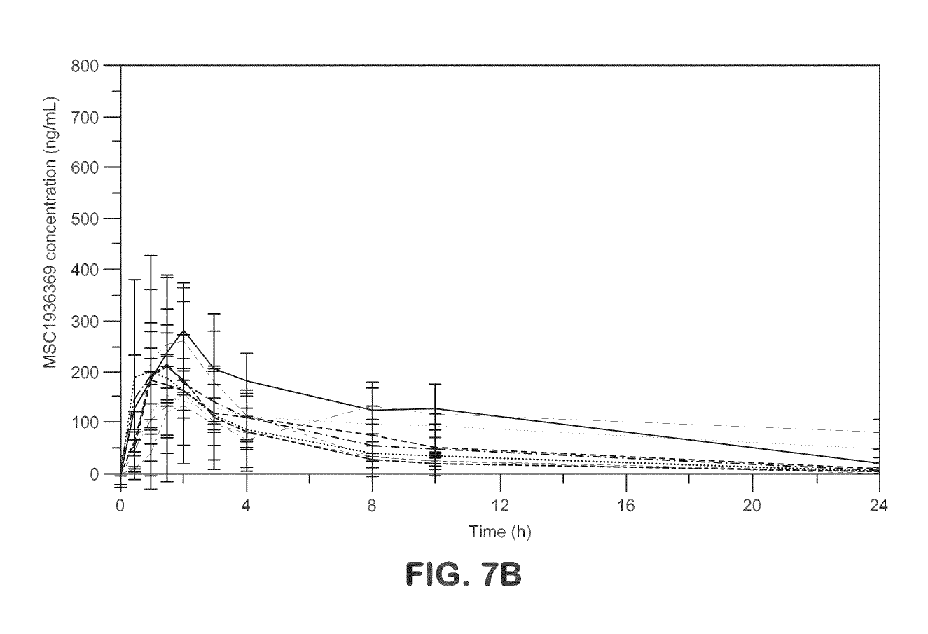

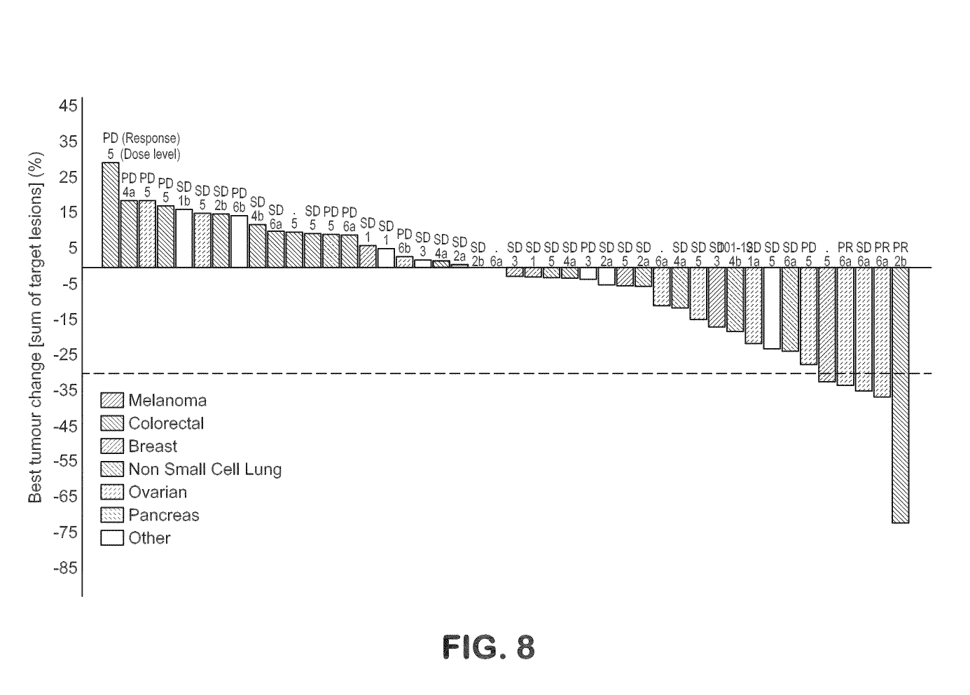

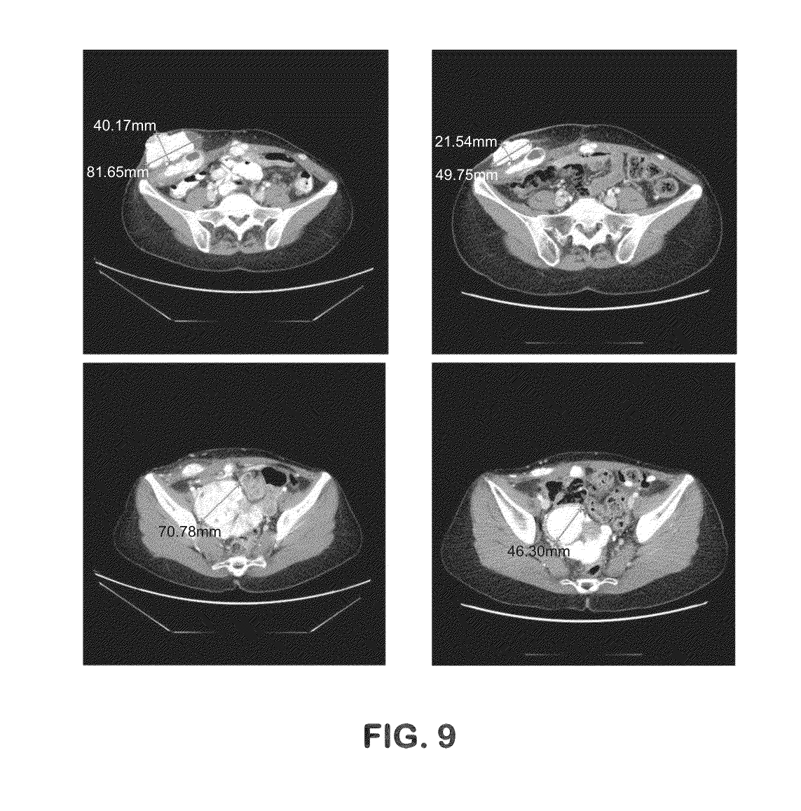

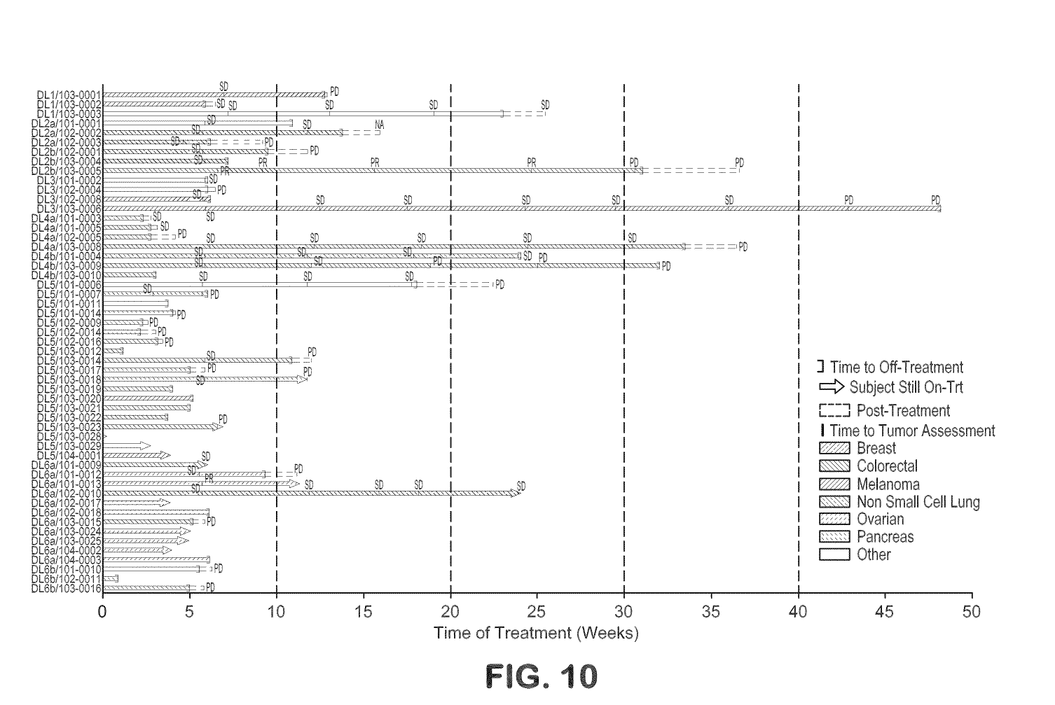

The results of the clinical trial are provided in Tables 10-11 and FIGS. 7 to 10. Of 64 patients evaluated as of 23 Feb. 2013, the median age was 58.5 years (range 26-82) and 54% had an ECOG PS of 1. The most common primary tumor types were: colorectal (CRC, n=22), ovarian cancer (n=13), pancreatic and non-small cell lung cancer (NSCLC, n=10 and 7 each). Dose escalation was stopped at DL6b as 2/3 patients experienced DLTs: both had grade 3 nausea and/or vomiting, leading to metabolic laboratory abnormalities. These adverse events (AEs) were reversible after treatment interruption and supportive care. The most frequent AEs were: dermatitis acneiform (72%), diarrhea (64%), fatigue (55%), nausea (48%) and vomiting (48%)). The median number of initiated cycles was 2 (range 1-16). There were three partial responses (KRAS mutated [mt] CRC with neuro-endocrine features, KRAS/BRAF wild-type [mt] low grade ovarian cancer, and KRAS /BRAF wild-type [wt] low grade ovarian cancer) and seven other patients had stable disease lasting ≧24 weeks (CRC [n=2, 1KRAS wt and 1 KRAS mt]; RAS mt NSCLC [n=2]; and BRAF wt melanoma, KIT mt soft palate cancer and PIK3CA mt bladder cancer [n=1, each]). The MTD was determined as DL6a (pimasertib 90 mg/SAR 245409 70 mg). DL5 was recommended as the phase II dose. Four disease-specific expansion cohorts (CRC, triple-negative breast cancer, NSCLC and melanoma), each to include 18 pts, are being treated at this dose. Dose escalation with twice-daily administration is ongoing. Preliminary PK and PD data showed no apparent drug-drug interaction.

TABLE 10
Patient distribution in dose levels

| | | | | | | No. of |
|---|---|---|---|---|---|---|
| | | | | | | sub- |
| | | Subjects | Time on exposure | | | jects |
| Dose | | enrolled | (weeks) | | | with |
| Level | Dose | n | Median | Min; | Max | DLTs |
| 1 | Compound (1) 15 mg + | 3 | 12.4 | 5.6; | 22.7 | — |
| | Compound (2) 30 mg | | | | | |
| 2b | Compound (1) 15 mg + | 3 | 9.4 | 7.1; | 31.0 | — |
| | Compound (2) 50 mg | | | | | |
| 2a | Compound (1) 30 mg + | 3 | 10.8 | 6.1; | 13.7 | — |
| | Compound (2) 30 mg | | | | | |
| 3 | Compound (1) 30 mg + | 4 | 6.1 | 6.0; | 48.1 | — |
| | Compound (2) 50 mg | | | | | |
| 4b | Compound (1) 30 mg + | 3 | 24.0 | 3.0; | 32.0 | — |
| | Compound (2) 70 mg | | | | | |
| 4a | Compound (1) 60 mg + | 4 | 2.7 | 2.3; | 33.4 | — |
| | Compound (2) 50 mg | | | | | |
| 5a# | Compound (1) 60 mg + | 19 | 3.7 | 0.1; | 18.0 | — |
| | Compound (2) 70 mg | | | | | |
| 6b | Compound (1) 60 mg + | 3 | 5.0 | 0.9; | 5.6 | 2* |
| | Compound (2) 90 mg | | | | | |
| 6a | Compound (1) 90 mg + | 11 | 5.9 | 3.4; | 24.0 | — |
| | Compound (2) 70 mg | | | | | |
| | Overall | 53 | 5.6 | 0.1; | 48.1 | 2* |

*Grade 3 nausea, vomiting and hyponatremia (1 subject) and Grade 2 nausea and Grade 3 hypokalemia (1 subject)
#includes pts enrolled in disease specific exp cohorts
Database as of 23 Nov. 2012, 3 pts missing as data not entered: 1 from DL6a and 2 from BID DL1a

TABLE 11
Adverse events occuring in >20% of patients, dose levels 1-6

| | | TEAEs |
|---|---|---|
| | All TEAEs | Grades ≧ 3 |
| | N = 53 | N = 53 |
| | n(%) | n(%) |
| Skin rash^{#} | 42 (72) | 8 (14) |
| Diarrhea | 37 (64) | 2 (3) |
| Asthenia/fatigue | 32 (55) | 2 (3) |
| Nausea | 28 (48) | 2 (3) |
| Vomiting | 28 (48) | 1 (2) |
| Edema peripheral | 20 (34) | 1 (2) |
| Pyrexia/hyperthermia | 18 (31) | 0 |
| Decreased appetite | 16 (28) | 0 |
| Serous retinal detachment^{#} | 16 (28) | 0 |
| Visual disturbances | 16 (28) | 0 |
| Gastro-esophageal reflux | 14 (24) | 0 |
| disease | | |
| Anemia | 13 (22) | 2 (3) |
| Dyspnea | 12 (21) | 3 (5) |
| Hypokalamia | 12 (21) | 4 (7) |

^{#}TEAEs related to Compound (1) and/or Compound (2).
Data base as of 23 Feb. 2013

We claim:

1. A method of treating cancer in a human patient, said method comprising administering to the patient an effective amount of (a) 2-amino-8-ethyl-4-methyl-6-(1H-pyrazol-5-y1)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, wherein said cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

2. The method according to claim 1, wherein the cancer is relapsed or refractory.

3. The method according to claim 1, wherein said effective amount is clinically proven safe.

4. The method according to claim 1, wherein the method comprises at least one cycle, wherein the cycle is a period of 3 weeks, wherein for each cycle the 2-amino-8-ethyl-4-methyl-6-(1H-pyrazol-5-yl)pyrido[2,3-d]pyrimidin-7(8H)-one or pharmaceutically acceptable salt thereof is administered at a daily dose of about 30, 50, 70 or 90 mg and the N—((S)-2,3-dihydroxypropyl)-3-(2-fluoro-4-iodo-phenylamino)isonicotinamide or pharmaceutically acceptable salt thereof is administered at a daily dose of about 15, 30, 60 or 90 mg.

5. The method according to claim 4, wherein for each cycle the 2-amino-8-ethyl-4-methyl-6-(1H-pyrazol-5-yl)pyrido[2,3-d]pyrimidin-7(8H)-one or pharmaceutically acceptable salt thereof is administered at a daily dose of about 70 mg and the N—((S)-2,3-dihydroxypropyl)-3-(2-fluoro-4-iodo-phenylamino)isonicotinamide or pharmaceutically acceptable salt thereof is administered at a daily dose of about 60 mg.

6. The method according to claim 1, wherein the effective amount produces at least one therapeutic effect selected from the group consisting of reduction in size of a tumor, reduction in metastasis, complete remission, partial remission, stable disease, increase in overall response rate, or a pathologic complete response.

7. The method according to claim 1, wherein the effective amount achieves a synergistic effect in reducing a tumor volume in said patient.

8. The method according to claim 1, wherein the effective amount achieves tumor stasis in said patient.

9. A composition comprising an effective amount (a) 2-amino-8-ethyl-4-methyl-6-(1H-pyrazol-5-yl)pyrido[2,3-d]pyrimidin-7(8H)-one or a pharmaceutically acceptable salt thereof, and (b) N—((S)-2,3-dihydroxypropyl)-3-(2-fluoro-4-iodo-phenylamino)isonicotinamide or a pharmaceutically acceptable salt thereof, wherein said cancer is selected from the group consisting of (i) KRAS or NRAS mutated non small cell lung cancer (NSCLC), (ii) triple negative breast cancer (TNBC), (iii) dual KRAS and PIK3CA mutated colorectal cancer (CRC) and (iv) BRAF mutated melanoma after progression on BRAF inhibitors.

10. The composition according to claim 9, wherein the composition is for use in treating cancer in a human patient.

11. The composition according to claim 10, wherein the use comprises at least one cycle, wherein the cycle is a period of 3 weeks, wherein for each cycle the 2-amino-8-ethyl-4-methyl-6-(1H-pyrazol-5-yl)pyrido[2,3-d]pyrimidin-7(8H)-one or pharmaceutically acceptable salt thereof is administered at a daily dose of about 70 mg and the N—((S)-2,3-dihydroxypropyl)-3-(2-fluoro-4-iodo-phenylamino)isonicotinamide or pharmaceutically acceptable salt thereof is administered at a daily dose of about 60 mg.
